(19) United States
(12) Patent Application Publication
(10) Pub. No.: US 2025/0313850 A1
(43) Pub. Date: Oct. 9, 2025

(54) CONSTRUCTION METHOD OF BACTERIAL CELLULOSE (BC)-ENRICHED PLANT USING MULTI-GENE TANDEM AND USE THEREOF

(71) Applicant: Shanghai Academy of Agricultural Sciences, Shanghai (CN)

(72) Inventors: Yongsheng TIAN, Shanghai (CN); Yongdong DENG, Shanghai (CN); Quanhong YAO, Shanghai (CN); Rihe PENG, Shanghai (CN); Wenhui ZHANG, Shanghai (CN); Xiaoyan FU, Shanghai (CN); Jing XU, Shanghai (CN); Bo WANG, Shanghai (CN); Zhenjun LI, Shanghai (CN); Jianjie GAO, Shanghai (CN); Hongjuan HAN, Shanghai (CN); Lijuan WANG, Shanghai (CN); Yu WANG, Shanghai (CN); Zhihao ZUO, Shanghai (CN); Cen QIAN, Shanghai (CN)

(21) Appl. No.: 18/629,619

(22) Filed: Apr. 8, 2024

(51) Int. Cl. C12N 15/82; C12N 9/06

(52) U.S. Cl. CPC C12N 15/8246; C12N 9/0028; C12N 15/8205; C12N 2710/00043; C12Y 105/01019

(57) ABSTRACT

The present disclosure provides a construction method of a bacterial cellulose (BC)-enriched plant using multi-gene tandem and use thereof, and relates to the technical field of genetic engineering. In the present disclosure, an acsAB gene, an acsC gene, and an acsD gene are combined and then subjected to codon optimization according to a codon preference of the crop. Resulting optimized genes are fused with a 35S promoter and a nopaline synthase (NOS) terminator to obtain three gene expression cassettes. The three gene expression cassettes are ligated into a plant expression vector to obtain a multi-gene plant transformation vector containing the above three gene expression cassettes. The multi-gene plant transformation vector is then transformed into rice to obtain a transgenic rice plant capable of synthesizing the BC. It is determined that the transgenic rice plant has a BC content of 3.81%.

### REFERENCE TO SEQUENCE LISTING

A computer readable XML file entitled “GWP20240301763_seqlist”, that was created on Apr. 2, 2024, with a file size of about 34,878 bytes, contains the sequence listing for this application, has been filed with this application, and is hereby incorporated by reference in its entirety.

### TECHNICAL FIELD

The present disclosure belongs to the technical field of genetic engineering, and specifically relates to a construction method of a bacterial cellulose (BC)-enriched plant using multi-gene tandem and use thereof.

### BACKGROUND

Bacterial cellulose (BC) is synthesized by fermentation of microbial strains, and has raw materials that come from a wide range of sources. The BC was discovered early by British scientist Brown. While studying Acetobacter xylinus, he accidentally discovered a layer of solid gel material formed on the surface of a culture solution. After corresponding characterization, this material was determined to be a high-purity cellulose, namely the “BC”. The BC shows a similar structure to that of plant cellulose, but has quite different physical and chemical properties, and does not contain hemicellulose, pectin, and lignin.

BC has a simple production process that causes no pollution to the environment, and can form a natural 3D nanofiber interwoven structure. The BC exhibits inherent unique characteristics such as high crystallinity and mechanical strength, desirable biocompatibility, and degradability. As a sustainable functional biomaterial, the BC is widely used in food, medicine, chemical industry and other fields.

The excellent properties of BC provide materials for various fields. In traditional methods, monosaccharides are generally used as carbon sources to prepare media for synthesizing BC, but these methods have a high cost that is not conducive to large-scale development. Therefore, it has become an urgent problem to be solved to produce the BC with low cost and high efficiency.

### SUMMARY

An objective of the present disclosure is to provide a construction method of a BC-enriched plant using multi-gene tandem and use thereof. In the present disclosure, a high-level expression of the BC in plant straw not only improves a utilization value of the straw but also avoids environmental pollution caused by burning the straw, thereby greatly improving social and economic benefits.

The present disclosure provides a gene combination for synthesizing BC by tandem expression in a plant genome, where the gene combination includes an acsAB gene, an acsC gene, and an acsD gene.

Preferably, the acsAB gene has a nucleotide sequence shown in SEQ ID NO: 1, the acsC gene has a nucleotide sequence shown in SEQ ID NO: 3, and the acsD gene has a nucleotide sequence shown in SEQ ID NO: 5.

The present disclosure further provides a multi-gene expression cassette constructed using the gene combination, where the gene combination is subjected to codon optimization when the multi-gene expression cassette is constructed.

The present disclosure further provides a construction method of the multi-gene expression cassette, including: fusing a codon-optimized acsAB gene with a 35S promoter and a nopaline synthase (NOS) terminator into an acsABS gene expression cassette;

fusing a codon-optimized acsC gene with the 35S promoter and the NOS terminator into an acsCS gene expression cassette; and

fusing a codon-optimized acsD gene with the 35S promoter and the NOS terminator into an acsDS gene expression cassette.

Preferably, the codon-optimized acsAB gene has a nucleotide sequence shown in SEQ ID NO: 2, the codon-optimized acsC gene has a nucleotide sequence shown in SEQ ID NO: 4, and the codon-optimized acsD gene has a nucleotide sequence shown in SEQ ID NO: 6.

Preferably, the 35S promoter is derived from cauliflower mosaic virus (CaMV); and the NOS terminator is derived from Agrobacterium rhizogenes.

The present disclosure further provides a recombinant plant transformation vector including the multi-gene expression cassette.

The present disclosure further provides a construction method of the recombinant plant transformation vector, including the following steps: ligating the acsABS gene expression cassette, the acsCS gene expression cassette, and the acsDS gene expression cassette prepared by the construction method in sequence in an order of acsABS-acsCS-acsDS, and then inserting into a pCAMBIA1301 vector between restriction sites EcoRI and BamHI to obtain the recombinant plant transformation vector denoted as pCAMBIA1301-acsABS-acsCS-acsDS.

The present disclosure further provides use of the gene combination or the multi-gene expression cassette or the recombinant plant transformation vector in promoting production of BC by a crop.

Beneficial effects: the present disclosure provides a gene combination for synthesizing BC by tandem expression in a plant genome, where an acsAB gene, an acsC gene, and an acsD gene are combined and then subjected to codon optimization according to a codon preference of the target crop, such that three gene expression cassettes are constructed. The three gene expression cassettes are ligated in a tandem manner into a plant transformation vector to obtain a recombinant plant transformation vector. In an example of the present disclosure, codon-optimized genes are separately fused with the 35S promoter of CaMV and the NOS terminator of Agrobacterium rhizogenes to construct gene expression cassettes. The gene expression cassettes are then ligated to a plant expression vector in sequence to obtain a multi-gene plant transformation vector, which is transformed into rice to obtain a transgenic rice plant capable of synthesizing the BC. PCR verification shows that the exogenous genes are completely integrated into the rice genome, and a total cellulose content in straw of the transgenic rice plant is measured in accordance with GB/T 2677.10-1995. The results show that the transgenic rice plants have a total cellulose content of 65.13%, of which the BC content accounts for 3.81%. New rice germplasm for synthesizing the BC can be created using the scheme of the present disclosure to obtain a reactor of BC production, which greatly increases an added value of rice, especially rice straw, thereby creating excellent social and economic benefits.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can be derived from these accompanying drawings by those of ordinary skill in the art without creative efforts.

FIG. 1 shows a schematic structural diagram of the plant transformation vector for three genes (acsABS, acsCS, and acsDS) in the present disclosure;

FIGS. 2A-C show PCR electrophoresis patterns (M: 15,000 bp DNA marker; WT: wild-type rice; 1, 2, 3: transgenic rice) for three genes (FIG. 2A acsABS, FIG. 2B acsCS, and FIG. 2C acsDS) in the present disclosure; and

FIG. 3 shows a comparison of the total cellulose content.

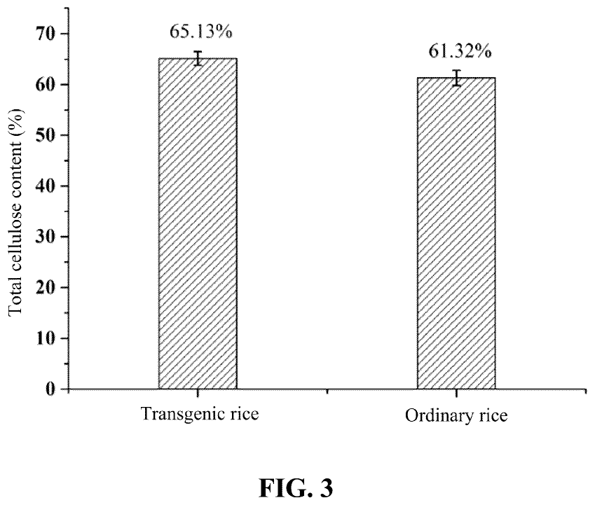

### DETAILED DESCRIPTION OF THE EMBODIMENTS

The present disclosure provides a gene combination for synthesizing BC by tandem expression in a plant genome, where the gene combination includes an acsAB gene, an acsC gene, and an acsD gene.

In the present disclosure, enzymes encoded by the three genes, acsAB, acsC, and acsD, are key enzymes for BC synthesis, where the enzyme encoded by the acsAB gene is a BC synthase, which helps to synthesize the BC; the enzyme encoded by the acsC gene participates in cell membrane channels to secrete cellulose; and the enzyme encoded by the acsD gene participates in crystallizing the cellulose into nanofibers. The acsAB gene has a nucleotide sequence preferably shown in SEQ ID NO: 1, the acsC gene has a nucleotide sequence preferably shown in SEQ ID NO: 3, and the acsD gene has a nucleotide sequence preferably shown in SEQ ID NO: 5.

The present disclosure further provides a multi-gene expression cassette constructed using the gene combination, where the gene combination is subjected to codon optimization when the multi-gene expression cassette is constructed.

In the present disclosure, codon optimization is preferably conducted based on the codon preference of a target plant. In the example, rice is used as the target plant for genetic transformation. Accordingly, the codon optimization is conducted according to the codon preference of rice by the following principles: (i) gene codons are optimized and a gene translation efficiency is improved according to rice codon preference; (ii) the recognition sites of commonly-used restriction endonucleases within the gene are eliminated to facilitate the construction of expression cassettes; (iii) inverted repeat sequences, stem-loop structures, and transcription termination signals are eliminated to balance GC/AT within the gene and improve RNA stability; (iv) proteins encoded by the gene are made conform to the N-terminal principle to improve the stability of the translated proteins; (v) the free energy of mRNA secondary structure is optimized to improve a gene expression efficiency. The codon-optimized acsAB gene (acsABS gene) has a nucleotide sequence shown in SEQ ID NO: 2, the codon-optimized acsC gene (acsCS gene) has a nucleotide sequence shown in SEQ ID NO: 4, and the codon-optimized acsD gene (acsDS gene) has a nucleotide sequence shown in SEQ ID NO: 6. In addition to the codon-optimized gene, each gene expression cassette also includes the 35S promoter and NOS terminator.

The present disclosure further provides a construction method of the multi-gene expression cassette, including: fusing a codon-optimized acsAB gene with a 35S promoter and a nopaline synthase (NOS) terminator into an acsABS gene expression cassette;
fusing a codon-optimized acsC gene with the 35S promoter and the NOS terminator into an acsCS gene expression cassette; and
fusing a codon-optimized acsD gene with the 35S promoter and the NOS terminator into an acsDS gene expression cassette.

In the present disclosure, there is no special limitation on a process of the fusing, which can be conducted according to conventional methods in the field; the 35S promoter is preferably derived from CaMV and has a nucleotide sequence preferably shown in SEQ ID NO: 7; the NOS terminator is preferably derived from Agrobacterium rhizogenes and has a nucleotide sequence preferably shown in SEQ ID NO: 8.

The present disclosure further provides a recombinant plant transformation vector including the multi-gene expression cassette.

In the present disclosure, a basic vector of the recombinant plant transformation vector preferably includes pCAMBIA1301, and the three gene expression cassettes are ligated into the pCAMBIA1301 in a tandem manner.

The present disclosure further provides a construction method of the recombinant plant transformation vector, including the following steps: ligating the acsABS gene expression cassette, the acsCS gene expression cassette, and the acsDS gene expression cassette prepared by the construction method in sequence in an order of acsABS-acsCS-acsDS, and then inserting into a pCAMBIA1301 vector between restriction sites EcoRI and BamHI to obtain the recombinant plant transformation vector denoted as pCAMBIA1301-acsABS-acsCS-acsDS.

The present disclosure further provides use of the gene combination or the multi-gene expression cassette or the recombinant plant transformation vector in promoting production of BC by a crop.

In the present disclosure, the crop preferably includes rice; in the examples, the recombinant plant transformation vector is transformed into the rice to obtain a transgenic rice plant capable of synthesizing the BC.

In order to further illustrate the present disclosure, the construction method of a BC-enriched plant using multi-gene tandem and the use provided by the present disclosure are described in detail below with reference to the accompanying drawings and examples, but the accompanying drawings and the examples should not be construed as limiting the protection scope of the present disclosure.

The test methods used in the examples are conventional molecular biology methods, unless otherwise specified; the materials and reagents used are commercially available, unless otherwise specified.

The formulas of each stock solution and each medium in the examples of the present disclosure are as follows:

### I. Formulas of Stock Solutions

MS max stock solution (10×): 16.5 g of NH₄NO₃, 19.0 g of KNO₃, 3.7 g of MgSO₄·7H₂O, 4.4 g of CaCl₂·2H₂O, and diluting with water to 1,000 mL.

MS min stock solution (100×): 0.083 g of KI, 0.62 g of H₃BO₄, 2.23 g of MnSO₄·2H₂O, 0.86 g of ZnSO₄·7H₂O, 0.025 g of Na₂MoO₄·2H₂O, 0.0025 g of CuSO₄·5H₂O, 0.0025 g of CoCl₂·2H₂O, diluting with water to 1,000 mL.

N6 max stock solution (10×): 28.3 g of KNO₃, 4.0 g of KH₂PO₄, 4.63 g of (NH₄)₂·SO₄, 1.85 g of MgSO₄·7H₂O, 1.66 g of CaCl₂)·2H₂O, diluting with water to 1,000 mL.

N6 min stock solution (100×): 0.08 g of KI, 0.16 g of H₃BO₄, 0.44 g of MnSO₄·2H₂O, 0.15 g of ZnSO₄· 7H₂O, diluting with water to 1,000 mL.

Fe²⁺-EDTA stock solution (100×): 2.78 g of FeSO₄·7H₂O and 3.73 g of Na₂EDTA·2H₂O, dissolving separately, mixing well, diluting with water to 1,000 mL.

Vitamin stock solution (100×): 0.1 g of Nicotinic acid, 0.1 g of vitamin B6 (Pyridoxine HCl, VB₆), 0.1 g of vitamin B1 (Thiaminc HCl, VB₁), 0.2 g of Glycine, 10 g of Inositol, diluting with water to 1,000 mL.

### II. Formulas of Media

Co-culture medium: 12.5 mL of the N6 max stock solution (10×), 1.25 mL of the N6 min stock solution (100×), 2.5 mL of the Fe²⁺-EDTA stock solution (100×), 2.5 mL of the vitamin stock solution (100×), 0.75 mL of 2 g/L dichlorophenoxyacetic acid (2,4-D), 0.2 g of Casein Enzymatic Hydrolysate, 5 g of Sucrose, 1.75 g of Agarose, adding water to 250 ml and adjusting to pH=5.6, melting in a microwave oven and adding 5 mL of 50% glucose and 250 μL of 20 g/L acetosyringone before use.

Selective medium: 25 mL of the N6 max stock solution (10×), 2.5 mL of the N6 min stock solution (100×), 2.5 mL of the Fe²⁺-EDTA stock solution (100×), 2.5 mL of the vitamin stock solution (100×), 0.625 mL of 2 g/L dichlorophenoxyacetic acid (2,4-D), 0.15 g of Casein Enzymatic Hydrolysate, 7.5 g of Sucrose, 1.75 g of Agarose, adding water to 250 mL and adjusting to pH=6.0, melting in oven and adding hygromycin and carbenicillin before use.

Predifferentiation medium: 25 mL of the MS max stock solution (10×), 2.5 mL of the MS min stock solution (100×), 2.5 mL of the Fe²⁺-EDTA stock solution (100×), 2.5 mL of the vitamin stock solution (100×), 0.5 mL of 6-benzylaminopurine (6-BA) 2 g/L, 0.5 mL of kinetin (KT) 2 g/L, 50 μL of indole acetic acid (IAA) 1 mg/mL, 0.15 g of Casein Enzymatic Hydrolysate, 7.5 g of Sucrose, 1.75 g of Agarose, adding water to 250 mL and adjusting to pH=5.9, melting in oven and adding hygromycin and carbenicillin before use.

Differentiation medium: 100 mL of the MS max stock solution (10×), 10 mL of the MS min stock solution (100×), 10 mL of the Fe²⁺-EDTA stock solution (100×), 10 mL of the vitamin stock solution (100×), 2.0 mL of 6-benzylaminopurine (6-BA) 2 g/L, 2.0 mL of kinetin (KT) 2 g/L, 0.2 mL of indole acetic acid (IAA) 1 mg/mL, 0.2 mL of naphthaleneacetic acid (NAA) 1 g/L, 1 g of Casein Enzymatic Hydrolysate, 30 g of Sucrose, 3 g of Phytagel, adding water to 1,000 mL and adjusting to pH=6.0, dispensing into vials.

Rooting medium: 50 mL of the MS max stock solution (10×), 5 mL of the MS min stock solution (100×), 10 mL of the Fe²⁺-EDTA stock solution (100×), 10 mL of the vitamin stock solution (100×), 20 g of agar powder (Sucrose), 3 g of Phytagel, adding water to 1,000 mL and adjusting to pH=5.8, dispensing into vials.

### Example 1 Construction of a Multi-Gene Plant Expression Vector

### 1. Optimized Synthesis of Three Genes

The acsAB gene (SEQ ID NO: 1), acsC gene (SEQ ID NO: 3), and acsD gene (SEQ ID NO: 5) of Acetobacter xylinus were used as templates, acsABS with a DNA sequence shown in SEQ ID NO: 2, acsCS with a DNA sequence shown in SEQ ID NO: 4, and acsDS with a DNA sequence shown in SEQ ID NO: 6 were synthesized based on the codon preference of rice, respectively, and their sequences were determined by sequencing.

The 35S promoter of CaMV was used as a template, the 35S promoter with a DNA sequence shown in SEQ ID NO: 7 was synthesized, cloned into a plasmid vector, and sequenced to determine its sequence.

The NOS terminator of Agrobacterium rhizogenes was used as a template, the NOS terminator with a DNA sequence shown in SEQ ID NO: 8 was synthesized, and sequenced to determine its sequence.

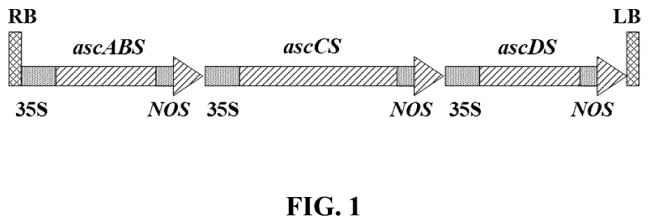

2. Construction of a multi-gene plant transformation vector: optimized three genes were fused with the 35S promoter and NOS terminator separately to construct three gene expression cassettes; the three gene expression cassettes were ligated in sequence (acsABS-acsCS-acsDS) using a ClonExpress MultiS multi-fragment one-step seamless rapid cloning kit (Norvizan) to form a complete sequence containing a multi-gene expression cassette; EcoRI and BamHI restriction sites were introduced at both ends of the complete sequence, and the complete sequence was determined using full nucleotide sequence analysis by Sangon Biotech (Shanghai) Co., Ltd. The correctly sequenced synthetic complete sequence was digested with EcoRI and BamHI and then ligated into a same enzyme-digested vector pCAMBIA1301 to obtain a multi-gene plant transformation vector containing the three genes, which was recorded as pCAMBIA1301-acsABS-acsCS-acsDS (FIG. 1).

The fusion of the gene with the 35S promoter and NOS terminator was completed using modified overlap extension PCR, where a specific reference for the modified overlap extension PCR was: (Rihe Peng, Aisheng Xiong, Quanhong Yao; A direct and efficient PAGE-mediated overlap extension PCR method for gene multiple-site mulagenesis, Applied Microbiology Biotechnology. 2006, 73:234-40), using Phanta MaxSuper-Fidelity DNA Polymerase provided by Vazyme Biotech Co., Ltd., which was suitable for high-fidelity amplification of long genes. The PCR amplification program included: pre-denaturation at 95° C. for 30 s; denaturation at 95° C. for 45 s, annealing at 56° C. to 72° C. for 45 s, extension at 72° C. for 5 min to 20 min (1,000 bp/min), amplifying for 25 to 35 cycles; final extension at 72° C. for 10 min.

### Example 2 Transformation of Rice

### 1. Obtaining and Identifying Transgenic Rice

### 1.1 Preparation and Electroporation of Agrobacterium

1) A single strain of Agrobacterium was selected and inoculated into 5 mL of LB liquid medium (rifampicin 50 μg/mL, chloramphenicol 100 μg/mL), and cultured at 28° C. and 250 rpm for 20 h.

2) 1 mL of a resulting bacterial solution was transferred into 20 mL to 30 mL of LB liquid medium (rifampicin 50 μg/mL, chloramphenicol 100 μg/mL), and cultured at 28° C. and 250 rpm for approximately 12 h, measured OD₆₀₀≈1.5.

3) The bacterial cells were collected by centrifugation at 8,000 rpm, 4° C. for 10 min, resuspended in Agrobacterium transformation permeate (5 wt % sucrose, 0.05 wt % Silwet L-77), and diluted to OD₆₀₀≈0.8.

4) The bacterial cells were fully resuspended in 0.5 times a volume of sterile water pre-cooled at 4° C., centrifuged at 3,500 g for 10 min, and the supernatant was carefully discarded. The bacterial cells were fully resuspended in 1 mL to 2 mL of sterile 10% glycerol pre-cooled at 4° C., and an obtained bacterial solution could be used immediately or frozen at −70° C.

5) 40 μL of the bacterial solution and (1-2) μL of plasmid DNA (0.4 pg to 0.3 μg) were added to 1.5 mL of eppendorf, mixed well in a 0.2 cm diameter electroporation cup, and placed on ice for about 1 min. The electroporation parameters were set to 25 μF, 2.5 kV/cm, and 400Ω. The electroporation was conducted for 4 msec to 5 msec.

6) After electroporation transformation, 1.0 mL of expression medium was added to the bacterial cells, incubated at 29° C. for 1 h, and spread on a YEB plate with antibiotics (rifampicin 50 μg/mL+kanamycin 50 μg/mL). The plant expression vector pCAMBIA1301-acsABS-acsCS-acsDS was electroporated into an Agrobacterium tumefaciens strain EHA105 using the above electroporation procedure to obtain a modified Agrobacterium tumefaciens strain EHA 105 (pCAMBIA1301-acsABS-acsCS-acsDS).

1.2 Agrobacterium Infection and Co-Culture with Rice Callus

### 1) Induction of Rice Callus:

The mature seeds (shelled) of Zhonghua 11 rice were soaked in 70% ethanol for 1 min, rinsed with sterilized water, then soaked in 2% sodium hypochlorite for 20 min to allow disinfection, and rinsed repeatedly with sterilized water. The seeds were placed in a sterile petri dish on a clean table, dried with sterilized filter paper, then the mature embryos were peeled off using a scalpel, and inoculated onto the medium with the scutellum facing up. The embryos were placed in a callus induction medium (including MS 4.4 g/L, 2,4-D 2.5 mg/L, casein 600 mg/L, sucrose 30 g/L, and phytagel 5 g/L, adjusted to pH=5.8 with KOH, and subjected to high-pressure sterilization) to allow callus culture, and incubated in the dark at 25° C. to 28° C. for 8 d to 10 d to induce callus tissue. When the germs grew to 1 cm, the scutellum calli with high quality were strictly selected on NBD2 medium (including NB and 2,4-D 2 mg/L), and incubated in the dark at 25° C. to 28° C. for 4 d to 7 d to allow subculture.

2) Culture of Agrobacterium tumefaciens for Infection:

Agrobacterium EHA105 with the plant expression vector (acsABS-acsCS-acsDS) was selected from the YEB plate, inoculated into 5 mL of YEB liquid medium containing 50 mg/L kanamycin, and incubated on a shaker at 200 rpm and 28° C. until OD₆₀₀=0.5, and then re-inoculated into fresh YEB medium at a ratio of 1:100 to allow shaking culture (the antibiotics contained in YEB medium and culture conditions were the same as before). At OD₆₀₀=0.5, the bacterial solution was centrifuged at a relative centrifugal force of 6,000×g for 10 min at 4° C., Agrobacterium cells were collected and resuspended with ⅔ MS+⅓YEB until OD₆₀₀=0.5 for later use.

3) Co-Culture of Agrobacterium tumefaciens:

The vigorously growing rice embryogenic callus was cut into small pieces of 2 mm and placed in a sterilized petri dish. Agrobacterium tumefaciens bacterial solution with OD₆₀₀=0.5 containing 100 μmol/L acetosyringone was added to allow soaking for 25 min. The bacterial solution on the surface was removed with sterile filter paper, the rice callus tissue was inoculated on the co-culture medium, and incubated in the dark at 28° C. for 2 d to 3 d.

### 4) Rice Callus Differentiation and Plant Regeneration

The rice calli after co-culture were collected, washed 3 times with sterile water containing 500 mg/L cephalosporin, and excess water was absorbed.

The calli were transferred to a screening medium (including NB medium, 2,4-D 0.5 mg/L, cefotaxime 600 mg/L, and hygromycin 25 mg/L) and cultured for 12 d to 15 d, where dark culture was conducted for the first 6 d to 7 d and the light culture was conducted for the last 8 d to 9 d at a light intensity of (45-55) mmoL·m⁻²·S⁻¹. The calli that had differentiated adventitious buds were transferred to the differentiation medium RE2-H and cultured for 15 d to make the adventitious buds grow into seedlings of 2 cm to 4 cm. The differentiation medium RE2-H included: MS, sucrose 30 g/L, sorbose (10-20) g/L, casein enzymatic hydrolysate 500 mg/L, 6-benzylaminopurine 1 mg/L, naphthylacetic acid 0.5 mg/L, kinetin 0.5 mg/L, zeatin 0.2 mg/L, hygromycin 50 mg/L, and agarose 8 mg/L, pH=5.8.

These seedlings were transferred to the rooting medium. After 2 weeks, they took root and were transplanted into long test tubes for 20 d to 30 d. The larger rice plants were transplanted to the field of Baihe Transgenic Base of Shanghai Academy of Agricultural Sciences.

### 2. Identification of Transgenic Rice

### 2.1 PCR Detection of Genomic DNA in Transformed Plant

Genomic DNA was extracted from transgenic rice leaves transplanted to the field using SDS method and used as a template, and the exogenous genes acsABS, acsCS, and acsDS were detected using PCR amplification. The primers were as follows:

acsABS:
F (SEQ ID NO: 9):
5′-TAC, AGT, GTC, ACC, TAC, CCC, TTC-3′;
R (SEQ ID NO: 10):
5′-TTG, TCC, CAA, GCA, GAC, CCT, GCA-3′.
SacsCS:
F (SEQ ID NO: 11):
5′-GAA, GAA, CGT, TGA, TGA, ACA, CAA-3′;
R (SEQ ID NO: 12):
5′-TGT, GTG, ATT, GAT, ATT, CAC, CTT-3′.
SacsDS:
F (SEQ ID NO: 13):
5′-GAA, GTT, TGA, GAG, AGA, ACC, CTT-3′;
R (SEQ ID NO: 14):
5′-GAC, CTT, CAA, GGA, CAG, GAG, CAA-3′.

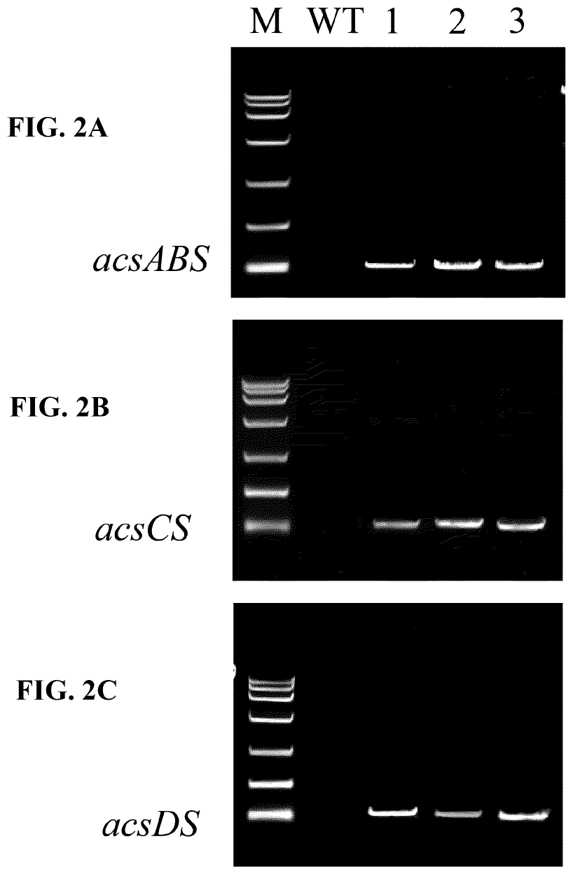

PCR amplification program included: pre-denaturation at 95° C. for 30 s; denaturation at 95° C. for 45 s, annealing at 56° C. to 72° C. for 45 s, extension at 72° C. for 5 min to 20 min (1,000 bp/min), amplifying for 25 to 35 cycles; final extension at 72° C. for 10 min. The results were shown in FIG. 2.

It was seen from FIG. 2 that all three transgenic plant lines could amplify the above three genes, indicating that the exogenous genes were completely integrated into the rice genome.

### Example 3 Detection of BC Content in Transgenic Rice Plant

The rice seeds harvested in Example 2 were germinated normally, and the rice straw was harvested when the rice matured, and then a total cellulose content was measured in accordance with GB/T 2677.10-1995. The measurement data showed that the transgenic rice had a total cellulose content of 65.13%, while the wild-type rice (control) had a total cellulose content of 61.32%. The above results indicated that the excess total cellulose in transgenic rice was BC, with a content of 3.81%.

### Example 4 Research on the Performance of Transgenic Rice Straw Used in Papermaking

The following experiments were completed at China Pulp and Paper Research Institute Co., Ltd.

1. Rice straw pulping: an appropriate amount of cut rice straw was cooked in a 20 L electric rotary cooking pot in the laboratory. The cooking conditions included: NaOH (calculated as Na₂O) 14%, anthraquinone 0.1%, solid-liquid ratio 1:5, maximum temperature 160° C., idling for 30 min, heating for 90 min, and holding temperature for 60 min. The cooked slurry was collected, the coarse slurry yield after washing was detected, and the fine slurry yield after screening was detected.

2. Analysis of basic fiber properties: according to GB/T 10336-2002 “Pulps—Determination of fiber length by automated optical analysis (Polarized light method)”, L&W fiber analyzer was used for detection.

3. Physical performance test of handsheets: the slurry was diluted to a certain concentration and handsheets were made in an automatic paper former. The handsheets had a quantitative value (60±3 g/m²), and the physical properties of the handsheet were measured in accordance with the corresponding national standards with a relative humidity (50±2) % at a temperature (23±1)° C. under a standard atmospheric pressure.

As shown in Table 1, the paper made using BC-enriched transgenic rice straw was superior to that made from wild-type rice straw in all aspects of performance, especially in tensile index and bursting index. The above results proved that the BC-enriched transgenic rice straw in the present disclosure could improve the performances of paper to a certain extent.

TABLE 1
Paper performance comparison

| | | | | | Wild-type | Transgenic |
|---|---|---|---|---|---|---|
| Item | | | | Unit | rice straw | rice straw |
| Slurry yield | Coarse slurry yield | | | % | 43.12 | 45.95 |
| | Fine slurry yield | | | % | 42.84 | 45.67 |
| Physical | Quantitation | | | g/m² | 62.0 | 62.9 |
| properties of | Thickness of uncompacted | | | cm³/g | 1.52 | 1.61 |
| handsheets | layer | | | | | |
| | Tensile index | | | N · m/g | 84.4 | 91.2 |
| | Elongate | | | % | 2.05 | 2.18 |
| | Bursting index | | | kPa · m²/g | 5.38 | 5.69 |
| Fiber basic | Length-weight average fiber | | | mm | 0.559 | 0.583 |
| performance | length | | | | | |
| analysis | Length-weight average fiber | | | μm | 13.5 | 13.4 |
| | width | | | | | |
| | Length | 0-0.2 | mm | % | 34.5 | 33.2 |
| | distribution | 0.2-0.5 | mm | | 31.2 | 30.1 |
| | | 0.5-0.8 | mm | | 17.0 | 15.0 |
| | | 0.8-1.1 | mm | | 8.7 | 10.2 |
| | | 1.1-1.4 | mm | | 4.5 | 4.7 |
| | | 1.4-1.7 | mm | | 1.2 | 1.9 |
| | | 1.7-2.0 | mm | | 0.8 | 1.6 |
| | | 2-3 | mm | | 0.9 | 1.8 |
| | | 3-5 | mm | | 1.1 | 1.6 |
| | | 5-7.5 | mm | | 0.1 | 0 |

| | | | |
|---|---|---|---|
| Fine fiber content | % | 34.3 | 33.2 |
| Average kink angle | ° | 49.664 | 48.235 |
| Number of kink angles | angles/mm | 0.400 | 0.398 |
| Number of kink angles | angles/fiber | 0.271 | 0.272 |
| Average kink index | / | 0.940 | 0.913 |

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A gene combination for synthesizing bacterial cellulose (BC) by tandem expression in a plant genome, wherein the gene combination comprises an acsAB gene, an acsC gene, and an acsD gene.

2. The gene combination according to claim 1, wherein the acsAB gene has a nucleotide sequence shown in SEQ ID NO: 1, the acsC gene has a nucleotide sequence shown in SEQ ID NO: 3, and the acsD gene has a nucleotide sequence shown in SEQ ID NO: 5.

3. A multi-gene expression cassette constructed using the gene combination according to claim 1, wherein the gene combination is subjected to codon optimization when the multi-gene expression cassette is constructed.

4. The multi-gene expression cassette according to claim 3, wherein the acsAB gene has a nucleotide sequence shown in SEQ ID NO: 1, the acsC gene has a nucleotide sequence shown in SEQ ID NO: 3, and the acsD gene has a nucleotide sequence shown in SEQ ID NO: 5.

5. A construction method of the multi-gene expression cassette according to claim 3, comprising: fusing a codon-optimized acsAB gene with a 35S promoter and a nopaline synthase (NOS) terminator into an acsABS gene expression cassette;
fusing a codon-optimized acsC gene with the 35S promoter and the NOS terminator into an acsCS gene expression cassette; and
fusing a codon-optimized acsD gene with the 35S promoter and the NOS terminator into an acsDS gene expression cassette.

6. The construction method according to claim 5, wherein the acsAB gene has a nucleotide sequence shown in SEQ ID NO: 1, the acsC gene has a nucleotide sequence shown in SEQ ID NO: 3, and the acsD gene has a nucleotide sequence shown in SEQ ID NO: 5.

7. The construction method according to claim 5, wherein the codon-optimized acsAB gene has a nucleotide sequence shown in SEQ ID NO: 2, the codon-optimized acsC gene has a nucleotide sequence shown in SEQ ID NO: 4, and the codon-optimized acsD gene has a nucleotide sequence shown in SEQ ID NO: 6.

8. The construction method according to claim 6, wherein the codon-optimized acsAB gene has a nucleotide sequence shown in SEQ ID NO: 2, the codon-optimized acsC gene has a nucleotide sequence shown in SEQ ID NO: 4, and the codon-optimized acsD gene has a nucleotide sequence shown in SEQ ID NO: 6.

9. The construction method according to claim 5, wherein the 35S promoter is derived from cauliflower mosaic virus (CaMV); and the NOS terminator is derived from Agrobacterium rhizogenes.

10. The construction method according to claim 6, wherein the 35S promoter is derived from cauliflower mosaic virus (CaMV); and the NOS terminator is derived from Agrobacterium rhizogenes.

11. A recombinant plant transformation vector comprising the multi-gene expression cassette according to claim 3.

12. The recombinant plant transformation vector according to claim 11, wherein the acsAB gene has a nucleotide sequence shown in SEQ ID NO: 1, the acsC gene has a nucleotide sequence shown in SEQ ID NO: 3, and the acsD gene has a nucleotide sequence shown in SEQ ID NO: 5.

13. A method for constructing a recombinant plant transformation vector, said method comprising the following steps: ligating the acsABS gene expression cassette, the acsCS gene expression cassette, and the acsDS gene expression cassette prepared by the construction method according to claim 5 in sequence in an order of acsABS-acsCS-acsDS, and then inserting into a pCAMBIA1301 vector between restriction sites EcoRI and BamHI to obtain the recombinant plant transformation vector denoted as pCAMBIA1301-acsABS-acsCS-acsDS.

14. The construction method according to claim 13, wherein the acsAB gene has a nucleotide sequence shown in SEQ ID NO: 1, the acsC gene has a nucleotide sequence shown in SEQ ID NO: 3, and the acsD gene has a nucleotide sequence shown in SEQ ID NO: 5.

15. The construction method according to claim 13, wherein the codon-optimized acsAB gene has a nucleotide sequence shown in SEQ ID NO: 2, the codon-optimized acsC gene has a nucleotide sequence shown in SEQ ID NO: 4, and the codon-optimized acsD gene has a nucleotide sequence shown in SEQ ID NO: 6.

16. The construction method according to claim 14, wherein the codon-optimized acsAB gene has a nucleotide sequence shown in SEQ ID NO: 2, the codon-optimized acsC gene has a nucleotide sequence shown in SEQ ID NO: 4, and the codon-optimized acsD gene has a nucleotide sequence shown in SEQ ID NO: 6.

17. The construction method according to claim 13, wherein the 35S promoter is derived from cauliflower mosaic virus (CaMV); and the NOS terminator is derived from Agrobacterium rhizogenes.

18. The construction method according to claim 14, wherein the 35S promoter is derived from cauliflower mosaic virus (CaMV); and the NOS terminator is derived from Agrobacterium rhizogenes.

19. A method for promoting production of BC by a crop, comprising using the gene combination according to claim 1.

20. The method for promoting production of BC by a crop according to claim 19, wherein the acsAB gene has a nucleotide sequence shown in SEQ ID NO: 1, the acsC gene has a nucleotide sequence shown in SEQ ID NO: 3, and the acsD gene has a nucleotide sequence shown in SEQ ID NO: 5.


SEQUENCE LISTING (14 sequences)

SEQ ID NO: 1 - DNA, 4653 bp, synthetic construct
  source 1..4653: mol_type = other DNA; note = acsAB gene
atgccagaggttcggtcgtcaacgcagtcagagtcaggaatgtcacagtggatggggaaaattctctccattcgcggtgctgggctgattattggtgtttttggcctgtgtgcgctgattgcggctacgtccgtgaccctgccgccagaacagcagttgattgtggcatttgtatgtgtcgtgatcttttttattgtcggtcataagcccagccgtcggtcccagattttccttgaagtgctgtcagggctggtttcgctgcgctatctgacatggcgcctgacggaaacgctttcattcgatacatggttgcagggtctgcttgggacaatgcttctggtggcggaactttacgccctgatgatgctgttcctcagctacttccagacgatcgcgccgctgcatcgtgcgcctctgccgctgccgccgaaccctgacgaatggcccacggtcgatatcttcgtcccgacctataacgaagaactgagcattgtccgcctgacggtgctgggatcactggggattgactggccaccggaaaaggtgcgggttcatatccttgatgacggccgtcgtcctgaattcgccgcctttgccgctgaatgtggcgcgaattatatcgcccgcccgacgaacgaacatgcaaaggccggtaatcttaactatgccattggtcataccgatggtgattacatcctgatctttgactgcgaccacgtcccgacccgcgccttcctgcagttgacaatgggctggatggtcgaagacccgaagatcgcgctgatgcagaccccgcatcacttctattcccccgacccgttccagcggaacctgtcggctggttatcgtaccccgcccgagggcaacctgttttatggcgtggtgcaggatggcaacgatttctgggatgcgaccttcttttgcgggtcatgtgcaatcctgcgtcgcacggcgattgagcagatcggcggctttgcgacccagaccgtgaccgaagacgcgcataccgcactcaagatgcagcgtctgggctggtccacggcctatctgcgtatcccgcttgccggtggtctcgcgacggaacgcctgatcctgcatatcggacagcgcgtgcgctgggcgcgtgggatgctgcagatcttccgcatcgacaatcctctgttcgggcgtggcctgtcatgggggcagcggctttgttacctgtcggccatgacgtcgttcctgttcgctgtcccgcgcgtcatcttcctgagctccccgctggcgttcctgttctttgggcagaacatcattgccgcgtcgccgctcgcgctgctggcctatgccatcccgcatatgttccacgccgtcggcacggcgtcgaagatcaacaagggctggcgctactccttctggagtgaggtctatgaaaccaccatggcgctgttcctggtgcgcgtgacgattgtcaccctgctcagcccttcacgtgggaagttcaacgtgacggacaagggcgggttgcttgaaaaaggttatttcgaccttggcgccgtctatccgaacatcatccttggcctgatcatgttcggcggtctggcgcgtggtgtctatgaactgtctttcggccatctcgaccagatcgccgaacgtgcctacctgctgaactccgcttgggcgatgctcagcctcatcatcatccttgcggccatcgccgtggggcgtgaaacacagcagaaacgcaacagtcatcgcatccccgcaaccatcccggtggaagtggcgaatgccgatgggtccatcatcgtgacgggcgtgaccgaggacctgtccatgggtggggccgcggtaaagatgtcatggcctgcgaagctgtcggggccgacgccggtttatatccgtactgtccttgatggagaggaactgatcctgcccgccaggatcatccgtgctggcaacgggcgggggatcttcatctggacgattgataacctgcagcaggaattctcggttatccgcctggtgttcggccgtgccgacgcatgggttgactggggcaattacaaggccgaccgcccgctgctcagcctcatggacatggttctcagcgtcaagggcctgttccgttcaagcggcgacatcgtccatcgcagttccccaaccaagccttcggctggcaatgccctgtctgacgatacgaacaacccgtcacgcaaggagcgtgtgctgaagggaaccgtgaaaatggtttcgcttctggcgctgctgacatttgcttcctcggcacaggcggcgtcagcgcccagggccgtcgcggcgaaggccccggcccatcagcccgaagcctctgacctgccgccgttgcccgcgctcctgccggccaccagcggtgcggcgcaggcgggttcgggcgatgccggcgccgatggacccggcagccccacgggccagcccctggcggccgacagtgccgatgcgctggtggaaaatgcggaaaatacgtccgatacggcgactgtccataattataccctcaaggatcttggcgccgcagggtccatcacaatgcgtggccttgccccgttgcaggggatcgagttcgggattccctctgaccagcttgtgacgtccgcgcgccttgtgctgtcgggttcgatgtcgcccaacctgcgtccggaaaccaattcggtcacaatgacgctgaacgagcagtatatcggcacgttgcgtcccgacccggcgcacccgacattcggccccatgtcgttcgagatcaacccgatcttcttcgtcagcggaaaccgtctgaacttcaacttcgcctccgggtcgaagggatgttcggacatcacgaacgatacgctgtgggccacgatctcgcagaactcgcagcttcagatcacgacaatcgcgctgccgccgcgtcgcctgctgtcacgtctgccgcagcccttctatgacaagaacgtgcgccagcatgttacggtcccgatggtgctggcgcagacctatgacccgcagatactcaagtccgcggggattctcgcttcgtggtttggcaagcagacggacttcctcggcgtgacgttcccggtgtcgtccaccatcccacagagtggcaacgccatcctgatcggcgtggccgatgaactgccgaccagcttcgggcggccgcaggtcaatggcccggcggttctggaactgccgaacccgtcggatgcaaatgccacgatcctggtggtgacggggcgtgaccgtgatgaggttattaccgcgagcaagggcatcgccttcgcgtctgctcccctgccgaccgacagccatatggatgtcgcgccggtcgatatcgccccgcgcaagcccaatgacgcgccatcttttatcgcgatggaccatccggtgcgctttggcgaccttgtaacggcaagcaagctacagggaaccggctttacgtccggtgtgctgtcggttccgttccgcattccgcccgacctttatacgtggcgtaaccgcccgtacaagatgcaggtacgtttccgttcccccgcgggggaggcgaaggatgtcgagaagtcacgtctcgatgtcgggatcaacgaggtttacctgcattcctatccgctgcgcgaaacgcatggcctgattggcgcggttttgcagggtgtcggccttgcccgccctgcaagcggcatgcaggtgcatgatctcgacgtaccgccgtggaccgtgttcgggcaggatcagttgaacttctactttgacgcgatgccccttgcgcgcggaatctgtcaaagtggcgcggcaaacaatgcattccatcttgggcttgacccggattccaccattgatttttcccgtgcccatcacatcgcccagatgcccaaccttgcctatatggcgacggtcggttttcctttcaccacctatgccgatctgtcgcagacggcggttgttctgcctgaacacccgaatgccgcgactgttggcgcctatcttgacctgatggggttcatgggcgcggcgacgtggtatccggttgcgggcgtggacattgtgtcggccgatcatgtcagtgacgttgcggaccgtaacctgctggtgatttcgacgctggcgacaagtggcgagatcgcgccgctgctgtcacgttcatcctacgaagtggcggatggtcatttgcgcacggtgtcgcatgcgtccgcgctggataacgcgatcaaggcggtcgacgatccgctgacggctttccgcgaccgtgacagcaagccgcaggatgtcgatacccccctgacgggtggtgtcggcgcgatgatcgaggcggaatcccccctgacggcgggccgcaccgttctggcgctgctgtcgtctgacggtgcggggctgaacaacctccttcagatgttgggggagcgcaagaagcaggcgaatatccagggggacctggttgttgcccatggcgaggacctgtcttcgtaccgcacttcgccggtctatacgatcggcaccctgccgctctggctgtggccggactggtatatgcacaacagaccggttcgtgtgctgcttgtcggcctgttgggatgtattctgatcgtcagtgttcttgcacgggctctggcgcggcatgcggcccggcgtttcaagcagcttgaggatgagaggcgcaagtcgtga

SEQ ID NO: 2 - DNA, 4653 bp, synthetic construct
  source 1..4653: mol_type = other DNA; note = codon-optimized acsAB gene
atgccagaggttcggagcagcacacagtcagagtcaggcatgtcacagtggatggggaagattctctccattcgcggtgctggactgattattggtgtttttggcctatgtgcactgattgcggctacgtccgtgaccctgccaccagaacaacagttgattgtggcattcgtctgtgtcgtgatcttttttattgtaggacataagcccagccgtcggtcccagattttccttgaagtactgtcagggctggtatcgctgcgctatctgacatggcgcctgacggaaacgctttcattcgatacatggttgcagggtctgcttgggacaatgcttctggtggcagagctttacgccctaatgatgctgttcctaagctactttcagacgatagcgccgctgcatcgtgcgcctctgccgctgccgccgaaccctgacgagtggcccacggtcgatatcttcgtcccgacatataacgaagagctgagcattgtccgcctaacggtgctgggatcactggggattgactggccaccggaaaaggtacgggttcatatccttgatgacggccgtcgtcctgagttcgccgccttcgccgctgaatgtggcgcgaactatatcgcccgcccgacgaacgaacatgcaaaggccggtaatctaaactatgccataggtcataccgatggtgactacatcctgatatttgattgcgaccacgtcccgacccgcgccttcctgcagttgaccatgggctggatggtcgaggacccgaagatagcgctgatgcagacaccgcatcacttctattccccagacccgttccagcggaatctgtcggctggttatcgtaccccgcccgagggcaacctattttatggagtggtgcaggatggcaacgatttctgggatgcgaccttcttttgcgggtcatgtgcaatcctgagacgcacggcgattgagcagatcggcggctttgcaacccagaccgtgaccgaagacgcgcataccgccctcaagatgcagcgtctaggctggtccacggcctacctgcgtatcccgctagccggtggtctagcgacagaacgcctgatcctgcatatcggacagcgcgtacgctgggcgcgtgggatgctgcagatcttcagaatcgacaatcctctattcggaagaggactgtcatgggggcagcggctttgttacctgtcggccatgacgtcgttcctgttcgctgtcccgagagtcatcttcctgagctccccgctggcgtttctgttctttgggcagaacatcattgcagcgtcgccactcgcactgctggcctatgccataccgcatatgttccacgccgtcggcacggcgtcgaagataaacaagggctggcgctactccttctggagtgaggtctatgaaaccaccatggcgctgttcctggtgcgcgtgacaattgtcaccctgctaagcccatcacgtgggaaattcaacgtgacggacaagggcgggttgcttgaaaaaggttacttcgatcttggcgccgtctatccaaatatcatccttggcctgatcatgtttggcggtctagcgcgtggtgtctatgaactgtctttcggccacctcgaccagatcgcagaacgtgcctacctgctgaactccgcttgggcgatgctaagtctcatcataatcctagcggccatcgccgtggggcgtgaaacacagcagaaacgcaatagtcatcgcatccccgcaaccatcccagtggaagtggcgaatgccgacggaagcatcatagtgacgggagtgaccgaagacctgtccatgggtggggccgcagtaaagatgtcatggcctgcgaagctatcggggccgacaccggtttatatccgtactgtccttgatggagaggaactgatcctgcccgccaggatcatccgtgctggcaacgggagagggatattcatctggacgatagataacctacaacaggaattctcggttatccgcctggtgttcggcagagccgacgcatgggttgactggggcaattataaggcagacagaccgctgctcagcctaatggacatggttctaagcgtcaagggcctgttccgtagcagcggcgacatcgtccaccgcagttccccaaccaagcctagcgctggcaatgccctgtctgatgacacgaacaacccatcacgcaaggagagagtgctaaagggcaccgtaaaaatggttagcctactggcgctactgacatttgcttcctcggcacaggcagcgtcagcgcccagggccgtcgcggcgaaggccccagcccatcagcccgaagcctctgacctgccgccattgcccgcgctactgccggcaaccagtggtgcggcgcaggcgggtagcggcgatgccggagcagatggacccggcagccccacgggccagccactggcagccgacagtgccgatgcgctggtggaaaatgcggagaatacgtccgatacggcgacagtccataattataccctcaaggatcttggagccgcagggtccatcacaatgcgtggccttgcaccgttgcaggggatcgagttcgggattccatctgatcaacttgtgacgagcgcgcgccttgtgctgtcgggtagcatgtcgcccaatctgcgtccggaaaccaattcggtcacaatgacgctgaacgagcaatatatcggcacgttgagaccagacccagcgcacccgacattcggacccatgtcgttcgaaatcaacccgatcttcttcgtcagcggaaaccgtctgaacttcaacttcgcctccgggtcgaagggatgttcggacataacgaacgatacgctgtgggccacgatcagccagaatagccaacttcagatcacgacaatcgcgctgccgccaagaagactgctaagccgtctgccgcagcccttctatgacaagaacgtgcgccagcatgttacggtcccgatggtgctggcgcaaacctatgatccgcagatactcaagtccgcggggattctcgcttcgtggtttggaaagcagacggactttctcggcgtgacgtttccagtgtcgtccacaatcccacagagtggcaacgcaatcctgatcggcgtggccgatgaactgccgaccagcttcgggcggccgcaagtaaacggcccagcggttctggaactgccgaacccgtcggatgcaaatgccacgatcctggtggtgacgggacgtgaccgtgatgaagttattaccgcgagcaagggaatagccttcgcgtctgctcccctgccgacagacagccatatggatgtcgcgccggtcgatatcgccccgagaaagcccaatgacgcaccatcttttatagcgatggaccacccggtgcgcttcggcgatcttgtaacggcaagcaagctccagggaacaggctttacgagcggtgtgctgtcggttccattccgcattccgcccgacctttatacgtggcgtaaccgcccgtacaagatgcaagtacgtttccgttcccccgcaggggaggcgaaggacgtcgagaagtcacgtctcgatgtcgggatcaacgaggtttacctgcacagctatccgctgcgcgagacgcacggcctaattggcgcagttttgcagggtgtcggcctagcccgccctgcaagcggcatgcaggtacatgatctcgacgtaccgccgtggaccgtgttcgggcaggaccagttgaacttctactttgacgcgatgccacttgcgcgcggaatctgtcaaagtggcgcggcaaacaatgcattccatcttgggcttgacccggattccaccattgatttttccagagcccatcacatcgcccagatgcccaaccttgcctatatggcgacagtcggtttcccattcaccacctatgccgatctatcgcagacggcggtagttctgcctgaacacccgaacgccgcgactgttggcgcctatctagatctgatggggttcatgggcgcggcgacatggtatccggttgcgggagtggacattgtatcggccgatcatgtaagtgacgttgcggaccgtaacctgctggtgatatcgacgctggcgacaagtggcgagatcgcaccgctgctgtcacgttcaagctacgaagtggcggatggacatttgcgcacggtgagccatgcgtccgcgctggataacgcgataaaggcggtcgacgatccgctgacggctttccgcgaccgtgacagcaagccgcaagacgtcgatacccccctgacgggtggtgtcggcgcgatgatcgaggcggaatcccccctgacggcgggccgcaccgttctggcgctgctgtcgtctgacggtgcggggctgaacaatctcctacagatgctaggggagcgcaagaaacaagcgaatatccagggagacctagttgttgcccatggcgaggacctgtctagctacagaactagcccggtctatacgatcggcaccctaccgctctggctgtggccggactggtatatgcataacaggccggttagagtgctacttgtcggcctgttgggatgtattctgatcgtcagtgttctagcccgggctctggcgcggcatgcagccagacgtttcaagcagcttgaggatgagaggcgcaagtcgtga

SEQ ID NO: 3 - DNA, 3909 bp, synthetic construct
  source 1..3909: mol_type = other DNA; note = acsC gene
gtgacccataaacgatatgcttcgtccctgtccgccggtcttctcgcaacgacctgcgtcgcaggtctgttgctgcaggcgaacggcgcacgggcacagcaggcggcagaggcgcaggccccggccagcagcaccaccatgatgcaagcagccaccgttgcccccgcccagagtgggcaggccgcggtggtgcagcggctggtgcagcaggcccgtttctggatgcagcagcaccagtatgaaaatgcgcgccagtccctgcagagtgctgcgcgacttgcaccggattccgttgatctgctggaggccgagggcgaataccagtcgcatatcggcaatcgtgatgccgcccttgatacgcagcgtcgcctgcatcaggccgcacctggcagcacgtatgaaagccagttgaacgacctgctgcatgaacaggcgatttcccagccggaccttgcgcatgcgcgctcgcttgccgcatccgggcacagcgatcaggcggtggaagcgtaccagcacctgttcaacggttcgacgcccacgccttcgctcgcggttgaatattaccagacgctggctggcgtctcggggcaggccggtacggcacaggatgggctgatccgtctggtcaaggccaatccttccgatttccgggcgcaactggcgcttgcacaggtcctgacctatcagcccggcacccggatggaggggctgcagcggcttcaggcgctccagaagtaccagtcttccgccccggtggaggctgcgacggcggaaaaatcatatcgccagacgctgtcatggttaccggttacacccgaaacactgccgttgatgcagaaatggctggatgcacatccatccgacagcgcattgcggacccatatggcagaaccggcaggcgggccgccggataaaggcgcgctggcgcggcaggacgggttcaaggcgttgaacgccggacgtctgtccgcagcccaggccgcgttccagagcgcactgaacctgaatgccaaggacggcgatgccctgggcggccttggccttgtcgccatgcgcgcgggccataacgaggaagcgcatcgttacctcgaagatgcgatcgcggctgatcccaagaatgcggcgcactggcgtccggcactggctggcatggccgtgggcgaggaatatggcagcgtccgtcgcctgatcgccagtggacagacacaggaagccgaacagcgcctgatgacgctggcgcgtcagcccggacagtccgagggcgcgacccttatgctggcggatttgcagcgcagcacgggccagaccggcgaggccgagcgcaattaccgggcgatcctggcgcgcaatggtgacaatcccatcgcactgatgggactggcccgtgtgctgatgggtgaaggccaggagagcgaagcgaacgccctgctgtcgcgccttggtggtcgctatagcgaccaggtacagcagatcgaggtttcgggcatcatggccgaagccgcccgcacgtcggattcagcgcagaaggtcagccttctgcggcaggccatgaccaaggccccggatgacccgtggttgcgcatcaaccttgccaatgcgctgcaacagcaaggtgacagcgcggaagcggccaatgtcatgcgtccgcttctgaccagcccgcgtacgccggccgattatcaggcggcgatcctgtatgcgtccggcaatggcaatgatacgctggcgcgccgtttgcttgcgggactgtcgccggatgactattccccggccatccgcaccattgccgatgaaatggcgatcaaggccgatctggccagccgcctttcgatggtttcgaatcccacgccgcttgtgcgcgaggctctggccgcgccggacccgacgggcgcgcgtggtgtggcggtggccgacctgttccggcagcgtggtgacatgctgcatgcgcatatggcgctgcgcatcgcctccacgcgcaatattgacctgacgaccgaacagcggctggcctatgccaccgaatatatgaagatcagcaatcccgttgccgcagcacgcctgcttgcaccgttgggcgatggcagcgggaccgcaaccggttccgcgatgtcgccggatcagcggcagacgctgatgcagttgcgcatgggtatttcggtggcacagtcggacctgctgaaccagcgtggcgatcaggcggcggcgtatgaccatcttgcgcccgcgttgcaggccgatccagaggcgacatcccccaagcttgcgttggcgcgactgtataacgggcgtggcaagtatgggcacgcgctggacatcgaccttgctgttcttcggcataatccgcaggaccttgatgcgcggcaggccgccgtgcaggccgcggcgaatgacggcaaggacaacctggccatgcaactggcgcaagatggggtccagcagtcgccgatggatgcgcgcagctggcttggcatggcggtggcggaccgcgctgtgggccatggcgaccggacgcttgctgacctgcgccgggcgtatgaattgcgcctgcagcagctcaagatcagtcggggtgatgccatcggcggtgatgaaacgcaggcgaccgcgccgccgacggccaatccgttccgtcgcgatgcatacgggcatgcgctgtcgttgggtgcgcccccgggtgagaacgggtacagcacggcaggaagcgtgcccgagatttcggaccagatgctgtcctccatcaacgggcagatccataccctgtccgaagacatggcgccgtctgttgatgccggccttggcttccgtgtccgttccggcaccccgggcatgggcgcgttgaccgaggcatccgtgccgatcgtcgggcgcatcccgctgcaggctggtacttcggcactgacattcacggccacgccaaccttcctgacgtcggggcacctgccgcagaccgggtatgatataccgcgtttcggcaccaatctgttcgcgctggaacggaacctgcagaaccagaacaacagcgcggaacatcgcatcaataccgatacgatcgggcgcgaggcaggtgtggcgcctgatgtccgctttgccaataactgggtcagtgccgatgtcggtgcctcgcctctgggctttacgctgccgaacgtgatcggtggtgttgaattcgccccgcgtgtcgggcctgtgaccttccgtgtcagtggggaacgccgctccattaccaacagcgtcctgtcctatggcgggatgaccgatgccctgaccggcaagaagtggggcggtgttgtcaccaaccacttccatggacaggtcgaggccacgctgggcaataccattgtgtatggtggcggtggttatgccatccagaccggccatcatgtccagagcaataccgaagtggaaggcgggctgggggcgaataccctggtttatcgcaaccgcaagcacgaagtgcgtgtcggtgtgaacctgacctatttcggctataagcataacgaggacttctatacttacgggcagggtggctacttctcgccgcagtcctattttgcggcaacggtgcccgtccggtattccgggcatagtggcctgtttgactgggatgtcaccgggtccatcggttatcagttgtttcatgaacacagttcggccttcttcccgaccaatcctgtgtatcaggcccttgcgaacgggctggcgggtgtatctactgctgaactatccttggaatcggccaggtatcccggcgatgatgtcggtagccttgttggtggtttcgatggcagggtgggttatcgcgtcagccattcgctgcgccttgatctgtccggtcgtttccagaaggctggaaactgggatgagggcggcgccatgatctcggctcactatcttattatggaccagtaa

SEQ ID NO: 4 - DNA, 3909 bp, synthetic construct
  source 1..3909: mol_type = other DNA; note = codon-optimized acsC gene
atgactcataaacgctatgcatcatcactttctgctggtcttcttgcaactacttgtgttgctggtctgcttctccaagcaaatggtgctcgtgcacaacaagctgctgaagcacaagcacctgcatcatcaactactatgatgcaagctgcaactgttgctcctgctcaatctggtcaagctgctgttgttcaacgtcttgttcaacaagcacgtttctggatgcaacaacaccaatacgaaaacgctcgtcaatcacttcaatctgctgctcgtcttgcacctgactctgtcgatctccttgaagctgaaggtgaatatcaatcacacattggtaatcgtgatgctgctcttgacactcaacgtcgccttcatcaagctgcacctggttctacttatgaatcacaactcaacgaccttctgcacgaacaagcaatctctcaacctgatcttgctcatgcacgctcacttgctgcatctggtcactctgaccaagctgtcgaagcataccaacacctcttcaatggttcaactccaactccatcactggctgtcgaatactatcaaactcttgctggtgtctctggtcaagctggtactgcacaagacggtctgatccgtcttgtcaaagcaaacccatctgacttccgtgctcaacttgctcttgctcaagtcctcacttaccaacctggtactcgcatggaaggtctccaacgcctgcaagcacttcagaagtaccaatcatctgcacctgttgaagctgcaactgctgagaaatcataccgtcagactctctcatggcttcctgtcactcctgaaactcttccactgatgcagaaatggcttgatgctcatccttctgactctgcactgcgtactcacatggctgaacctgctggtggtccacctgacaaaggtgctcttgcacgtcaagatggtttcaaagctctcaacgctggtcgtctctctgctgcacaagctgctttccaatctgcactcaacctcaacgccaaagatggtgatgctcttggtggtcttggtctcgtcgctatgcgtgctggtcacaacgaagaagcacatcgctaccttgaagatgctatcgctgctgaccctaagaacgctgcacactggcgtcctgcacttgctggtatggctgttggtgaagaatacggttctgtccgtcgcctgattgcttctggtcaaactcaagaagctgaacaacgccttatgactcttgctcgtcaacctggtcaatctgaaggtgctactctcatgcttgctgaccttcaacgctcaactggtcaaactggtgaagctgaacgtaactaccgtgcaatccttgcacgtaatggtgacaacccaattgcactcatgggtcttgcacgtgttcttatgggtgaaggtcaagaatctgaagccaatgcacttctctcacgccttggtggtcgttactctgaccaagtccaacagatcgaagtctctggtatcatggctgaagctgcacgcacctctgactctgcacagaaggtctcacttcttcgtcaagctatgactaaggcacctgacgacccttggcttcgcatcaaccttgcaaatgcacttcaacaacaaggtgactctgctgaagctgcaaacgtcatgcgtccactgctcacttcaccacgtactcctgctgactaccaagctgcaatcctctacgcatctggtaacggtaacgacactctcgcacgtcgccttcttgctggtctctctcctgatgactactcacctgcaatccgtaccatcgctgatgaaatggctatcaaagctgaccttgcttctcgtctctctatggtctccaatcctactcctcttgttcgtgaagctcttgctgcacctgatccaactggtgctcgtggtgttgctgttgctgatctcttccgtcaacgtggtgatatgcttcatgctcacatggcactgcgtatcgcatcaactcgcaacattgaccttactactgaacaacgccttgcatacgcaactgaatacatgaaaatctccaatcctgttgctgctgcacgccttctcgcaccacttggtgatggttctggtactgcaactggttctgctatgtctcctgatcaacgtcaaactcttatgcaacttcgtatgggtatctctgtcgctcaatctgaccttctcaaccaacgtggtgatcaagctgctgcatacgatcaccttgcacctgcactgcaagctgatcctgaagctacttcacctaaacttgcacttgcacgcctctacaacggtcgtggtaagtacggtcatgcacttgacatcgaccttgctgttcttcgccacaacccacaagacctcgacgcacgccaagctgctgttcaagctgctgctaacgatggtaaagacaaccttgcaatgcaacttgcacaagacggtgtccaacaatctcctatggatgctcgctcttggcttggtatggctgttgctgatcgtgctgttggtcatggtgatcgtactcttgctgatcttcgtcgtgcttacgaacttcgtcttcaacaactgaaaatctctcgtggtgatgcaatcggtggtgatgaaactcaagctactgcaccaccaactgccaatcctttccgtcgcgacgcttacggtcatgcactgtcactgggtgcaccacctggtgagaatggttactcaactgctggttctgttcctgaaatctctgatcagatgctgtcatcaatcaacggtcagatccatactctgtctgaagacatggctccttctgtcgatgctggtcttggtttccgcgtccgttctggtactcctggtatgggtgctcttactgaagcatctgttccaatcgttggtcgcattccactccaagctggtacttctgcactgaccttcactgcaactccaaccttcctcacctctggtcatcttccacagactggttacgacatcccacgcttcggtactaacctcttcgcacttgaacgcaaccttcagaaccagaacaactctgctgaacatcgcatcaacactgataccattggtcgcgaagctggtgttgctcctgatgttcgcttcgcaaacaactgggtctctgctgatgttggtgcatcaccactcggtttcacccttcctaacgtcattggtggtgtcgagttcgcaccacgtgttggtcctgtcaccttccgtgtctctggtgaacgccgttccatcactaactccgttctctcatacggtggtatgactgatgcactcactggtaagaaatggggtggtgttgtcaccaaccacttccatggtcaagtcgaagccactcttggtaacactatcgtctatggtggtggtggttacgcaatccaaactggtcatcatgtccaatccaacactgaagttgaaggtggtcttggtgccaacactcttgtctatcgcaaccgcaaacacgaagtccgtgttggtgtcaaccttacttactttggttacaaacacaacgaagacttctacacttacggtcaaggtggttacttctcaccacagtcctacttcgctgcaaccgttcctgttcgctactctggtcactctggtctgttcgattgggatgtcactggttcaatcggttatcaactgttccacgaacactcatctgcattcttcccaaccaaccctgtctatcaagcacttgcaaacggtcttgctggtgtctccactgctgaactctcactggaatctgcacgctaccctggtgatgacgttggttcacttgttggtggtttcgacggtcgcgttggttatcgcgtctcccactcacttcgccttgatctctctggtcgcttccagaaggctggtaattgggacgaaggtggtgccatgatctccgcacactacctgatcatggaccaataa

SEQ ID NO: 5 - DNA, 471 bp, synthetic construct
  source 1..471: mol_type = other DNA; note = acsD gene
atgacaatttttgagaaaaaaccggatttcaccctgtttcttcagaccctgtcatgggaaattgatgatcaggtcgggatcgaggtcaggaacgagctcctgcgtgaggtcggacggggcatgggcacgcgcatcatgccgccgccgtgccagaccgtggacaagctgcagatcgaactgaacgcgcttctggccctgatcggctggggcaccgtgacgctcgaactcctcagcgaggaccagtccctgcgcatcgtgcatgaaaacctgccgcaggttggcagcgcgggcgaaccttcgggcacgtggctggctccggtgctggaggggctgtatggccgctgggtgacgtcgcaggcgggtgcgtttggtgattatgtcgttacacgcgatgtggacgccgaggatctgaatgctgttccgcgtcagaccatcatcatgtacatgcgcgtgcgcagttccgcgacctga

SEQ ID NO: 6 - DNA, 471 bp, synthetic construct
  source 1..471: mol_type = other DNA; note = codon-optimized acsD gene
atgactatcttcgagaagaaacctgacttcactctcttccttcaaactctctcttgggaaatcgacgatcaagttggtatcgaagttcgtaacgaacttcttcgtgaagttggtcgtggtatgggtactcgtatcatgcctcctccttgtcaaactgttgacaaacttcaaatcgaacttaacgctcttcttgctcttatcggttggggtactgtcactcttgaacttctttctgaagatcaatctcttcgtatcgtccacgagaaccttcctcaagttggttctgctggtgaaccttctggtacttggcttgctcctgtccttgaaggtctctacggtcgttgggtcacttctcaagctggtgctttcggtgactacgtcgtcactcgtgatgtcgatgctgaagacctcaacgctgttcctcgtcaaactatcatcatgtatatgcgtgttcgttcttctgctacttaa

SEQ ID NO: 7 - DNA, 537 bp, synthetic construct
  source 1..537: mol_type = other DNA; note = 35S promoter
atggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggact

SEQ ID NO: 8 - DNA, 865 bp, synthetic construct
  source 1..865: mol_type = other DNA; note = NOS terminator
ccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcaaacttcggtactgtgtaatgacgatgagcaatcgagaggctgactaacaaaaggtatgcccaaaaacaacctctccaaactgtttcgaattggaagtttctgctcatgccgacaggcataacttagatattcgcgggctattcccactaattcgtcctgctggtttgcgccaagataaatcagtgcatctccttacaagttcctctgtcttgtgaaatgaactgctgactgccccccaagaaagcctcctcatctcccagttggcggcggctgatacaccatcgaaaacccacgtccgaacacttgatacatgtgcctgagaaataggcctacgtccaagagcaagtcctttctgtgctcgtcggaaattcctctcctgtcagacggtcgtgcgcatgtcttgcgttgatgaagctt

SEQ ID NO: 9 - DNA, 21 bp, synthetic construct
  source 1..21: mol_type = other DNA; note = forward primer for acsABS
tacagtgtcacctaccccttc

SEQ ID NO: 10 - DNA, 21 bp, synthetic construct
  source 1..21: mol_type = other DNA; note = reverse primer for acsABS
ttgtcccaagcagaccctgca

SEQ ID NO: 11 - DNA, 21 bp, synthetic construct
  source 1..21: mol_type = other DNA; note = forward primer for acsCS
gaagaacgttgatgaacacaa

SEQ ID NO: 12 - DNA, 21 bp, synthetic construct
  source 1..21: mol_type = other DNA; note = reverse primer for acsCS
tgtgtgattgatattcacctt

SEQ ID NO: 13 - DNA, 21 bp, synthetic construct
  source 1..21: mol_type = other DNA; note = forward primer for acsDS
gaagtttgagagagaaccctt

SEQ ID NO: 14 - DNA, 21 bp, synthetic construct
  source 1..21: mol_type = other DNA; note = reverse primer for ascDS
gaccttcaaggacaggagcaa